(19) United States
(12) Patent Application Publication
(10) Pub. No.: US 2025/0376626 A1
(43) Pub. Date: Dec. 11, 2025

(54) STEAM TREATMENT OF WASTE

(71) Applicant: SRU INNOVATIONS LTD, Staplefield (GB)

(72) Inventors: Alan Charles Pearce, Farringdon (GB); Natalie Adele Winter, Exwick (GB)

(73) Assignee: SRU INNOVATIONS LTD, Staplefield (GB)

(21) Appl. No.: 19/107,883

(22) PCT Filed: Aug. 3, 2023
(86) PCT No.: PCT/EP2023/071592

§ 371 Date: Feb. 28, 2025

(30) Foreign Application Priority Data
Sep. 2, 2022 (GB) 2212827.6

(51) Int. Cl. C10B 49/04; B09B 3/45; B09B 101/17; B09B 101/75; B09B 101/77; B09B 101/80; C07C 67/48; C10B 7/10; C10B 53/07

(52) U.S. Cl. CPC C10B 49/04; B09B 3/45; C07C 67/48; C10B 7/10; C10B 53/07; B09B 2101/17; B09B 2101/75; B09B 2101/77; B09B 2101/80

(57) ABSTRACT

The present invention relates to a method of treating waste material using superheated steam in an apparatus, the apparatus comprising: a treatment vessel comprising a treatment zone wherein at least one steam inlet is located at one end of the treatment zone and at least one steam outlet is located at the opposite end of the treatment zone; the method comprising: a. a loading step, comprising loading the waste material at a temperature of less than 50 degrees C. into the treatment zone; b. a treatment step comprising: i. feeding superheated steam at a temperature of from 300 to 800 degrees C. into the treatment zone through the at least one steam inlet and ii. removing steam and any gaseous reaction products through the at least one steam outlet; and c. a removal step comprising removing any remaining solid product from the treatment vessel after the treatment step wherein apart from the superheated steam, any additional heating applied in the treatment zone during the treatment step raises the temperature of the treatment zone by 100 degrees C. or less.

### FIELD OF THE INVENTION

The present invention relates to a method for decomposing and hydrolysing waste materials using steam to generate a range of useful products.

### BACKGROUND

The effective disposal of waste materials, in particular plastic, municipal solid waste, created by industrial, commercial and household activities is a key challenge for modern societies. Some of the most common methods for disposing of waste materials include landfill and incineration. However, both of these methods result in environmental pollution and mean that the useful chemicals contained in the waste materials cannot be recycled.

More recently, pyrolysis processes have been used to treat waste materials. Pyrolysis involves the thermal decomposition of materials at elevated temperatures in an inert atmosphere. Pyrolysis processes help to reduce the volume of the waste. However, pyrolysis has the disadvantage that high temperatures are required to treat the waste material and that treatment of mixed waste materials can be complicated. Moreover, the high temperatures employed in pyrolysis processes lead to the loss and breakdown of volatile components during the process. The end product of a pyrolysis process is generally a pyrolysis oil, which is a tar like substance from which it is difficult to separate useful chemicals.

Steam reforming is a method for producing syngas (hydrogen and carbon monoxide) by the reaction of hydrocarbons with water. Generally, methane is used as the feedstock for this reaction and the main purpose is hydrogen production. Steam reforming has recently been applied to waste, see for example US 20110204294A1, however, this process requires elevated temperatures and does not allow the separation and re-cycling of volatile components from the waste material.

There remains a need in the art for improved waste disposal processes for a variety of waste materials, which can operate at moderate temperatures and allow the separation and re-cycling of volatile components from the waste material. Moreover, there is a need for waste treatment processes which produce carbonaceous materials as their products which help lock-up carbon, preventing it from re-entering the atmosphere as carbon dioxide.

### SUMMARY OF THE INVENTION

The present inventors have developed a steam reforming process which helps to address the practical problems outlined above.

Accordingly, in a first aspect the present invention provides a method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
a loading step, comprising loading the waste material at a temperature of less than 50° C. into the treatment zone;
a treatment step comprising:
   i. feeding superheated steam at a temperature of from 300 to 800° C. into the treatment zone through the at least one steam inlet and
   ii. removing steam and any gaseous reaction products through the at least one steam outlet; and
a removal step comprising removing any remaining solid product from the treatment vessel after the treatment step
wherein apart from the superheated steam, any additional heating applied in the treatment zone during the treatment step raises the temperature of the treatment zone by 100° C. or less.

The method of treating waste material according to the present invention has a number of advantageous features.

Firstly, the method according to the present invention can repurpose a wide range of carbon-based materials, in particular, the process can be used for mixed waste materials meaning that there is no need to sort or clean the waste materials before reprocessing them, as is the case for other disposal methods. This means that the method according to the present invention is simpler than existing methods of waste re-processing and does not require the use of expensive sorting equipment. The method according to the present invention is also able to cope with considerable quantities of chemically inert material (such as sand, pieces of brick, silica or glass).

Secondly, the method according to the present invention is able to cope with problem materials such as tyres and polyvinyl chloride (PVC) and does not result in the production of chlorine gas or other gaseous chlorinated compounds or require the addition of additional components such as lime to soak up toxic chemicals.

Thirdly, the process allows valuable volatile chemicals to be stripped out of the waste material and then recondensed from the steam at the at least one steam outlet. This allows the effective recycling of valuable chemical components such as the plasticizer triethyl citrate which is found in tyres and other plastic materials.

Fourthly, the energy required to run the process in most cases can be derived from burning a fraction of the gaseous or solid products produced. This means that the method can be self-sustaining and that no additional fuel is required to run the method.

Fifthly, the method can be used to process electronic waste; following the reaction the solid product of the reaction can be treated to remove any carbonaceous material and fibreglass to give a concentrate consisting of metals, ceramics and semiconductors. This concentrate can then be separated in order to obtain the precious metals present in the electronic waste.

Sixthly, the solid product can be used to sequester carbon meaning that it is removed from the atmosphere permanently.

In a further aspect, the present invention provides a method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
a loading step, comprising loading the waste material at a temperature of less than 50° C. into the treatment zone;
a treatment step comprising:
   i. feeding superheated steam at a temperature of from 300 to 800° C. into the treatment zone through the at least one steam inlet and
   ii. removing steam and any gaseous reaction products through the at least one steam outlet so as to establish a temperature gradient across the treatment zone; and
a removal step comprising removing any remaining solid product from the treatment vessel after the treatment step.

Generally, the temperature gradient is established between the at least one steam inlet and between the at least one steam outlet.

In a further aspect, the present invention provides a solid product obtainable by the method according to the present invention.

In a further aspect, the present invention provides a gaseous reaction product obtainable by a method according to the present invention.

Within the meaning of this invention the “treatment zone” is defined as the section of the treatment vessel between the at least one steam inlet and the at least one steam outlet. Generally, the treatment zone is the area where the steam contacts the waste material.

Within the meaning of this invention the term “opposite end of the treatment zone” is intended to refer to the other end of the treatment zone, so that the at least one steam inlet and the at least one steam outlet define the boundaries of the treatment zone by their location in the treatment vessel.

Within the meaning of this invention a “continuous process” is defined as a process in which waste material is continuously provided to the treatment zone and the products are continuously removed.

By “mixed waste material” we mean any combination of different waste types. Generally, the waste material is derived from farming, industrial, commercial and household activities and is a solid waste material, a solid-slurry or a solid-containing waste material. Generally, the term “mixed waste material” refers to non-gaseous waste materials.

Without wanting to be bound by any theory, under some definitions the method according to the present invention is not a pyrolysis process.

The term “pyrolysis process” may refer to a process in which the waste stream is decomposed by heat and reacts only with components in the waste stream itself, with no additional reactants being added. In contrast, without wanting to be bound by any theory it is believed that the method according to the present invention may involve steam hydrolysis wherein the reaction is primarily between components of the waste stream and steam, with lysis of the waste being accomplished principally by H⁺ and OH⁻ ions rather than solely by the action of heat.

Throughout the application the term “recycling” is used to cover both recovery of a component from waste material and/or subsequent re-use of the recovered component.

Without wanting to be bound by any theory, the method according to the present invention is optionally not a fluidized bed process.

### Heating

In the method according to the present invention, the heat energy in the treatment zone is derived from the superheated steam.

Generally, it is expected that the method according to the present invention will be operated at normal ambient temperatures for example from about −10° C. (cold UK winter) to about 50° C. (extremely hot tropical summer). Optionally, the temperature of the environment (room or outside area) containing the apparatus will be from about −10° C. to about 50° C., for example the method may be conducted at normal UK outdoor temperatures (from about 0° C. to about 30° C.) or at room temperature (about 25° C.) if the method is carried out inside. Generally, the apparatus during the loading step is expected to be at a temperature of from about −10° C. to about 50° C.

The term “additional heating applied in the treatment zone” refers to any heating of the treatment zone apart from the superheated steam. This term, however, is not intended to encompass any heating required to heat the environment in which the method is being caried out to ambient temperature (about −10° C. to about 50° C.), for example central heating of a room.

Preferably any additional heating applied in the treatment zone during the treatment step raises the temperature of the treatment zone by 100° C. or less; Preferably, the additional heating applied in the treatment zone during the treatment step raises the temperature of the treatment zone by 70° C. or less, more preferably by 50° C., more preferably by 20° C. or less, more preferably by 10° C. or less.

Without being bound by any theory, for the scenario in which the temperature of the steam at the at least one steam inlet is 400° C., this may mean that additional heating is used to raise the temperature of the treatment zone to no more than 500° C., preferably to no more than 470° C., more preferably to no more than 450° C., more preferably to no more than 420° C., more preferably to no more than 410° C., wherein the temperature is the temperature of the steam at any point within the treatment zone at any time during the treatment step or the temperature of a probe at any point within the treatment zone at any time during the treatment step.

Alternatively, the degree of additional heating may be measured using the following method:
running the method with additional heating and measuring the temperature of a temperature probe in the treatment zone at the point where the waste is treated;
running the same method without additional heating and measuring the temperature of the temperature probe in the treatment zone at the point where the waste is treated;
   wherein the temperature is measured at the same location in the treatment zone and wherein the temperature is measured at the same time point after the beginning of the treatment step (the treatment step being the time a piece of waste material spends in the treatment zone) in both methods. Without being bound by any theory, it is preferred that the measurement is conducted once the treatment zone is at a steady state.

Preferably, when the temperature of the superheated steam at the at least one steam inlet is from 300 to 800° C. the maximum temperature in the treatment vessel is from 300 to 900° C.

Most preferably, no additional heating apart from the superheated steam is applied to the treatment zone during the treatment step. In this scenario all of the thermal energy provided to the treatment zone is from the superheated steam.

### Steam Inlet Temperature

In the method according to the present invention the superheated steam is fed into the treatment zone through the at least one steam inlet at a temperature of from 300 to 800° C. (wherein the temperature of the steam is measured at the at least one steam inlet).

At higher temperatures for example above about 650° C. the superheated steam will react with the carbon in the solid product forming hydrogen and carbon monoxide. Therefore, methods wherein the superheated steam at the at least one inlet is above about 650° C. can be used to produce hydrogen gas. Preferably, the superheated steam at the at least one inlet is above 750° C. to 800° C. for methods used to produce hydrogen gas. The temperature of the superheated steam is measured directly at the at least one steam inlet using any conventional means known in the art such as a thermocouple or thermometer.

Preferably, the method according to the present invention makes use of superheated steam at more moderate temperatures.

Without being bound by any theory processes carried out at lower temperatures lead to a solid waste product and are less energy intensive.

Preferably, the temperature of the superheated steam at the at least one inlet is from 400 to 600° C., more preferably from 400 to 550° C., most preferable from 400 to 500° C.

Optionally, the method according to the present invention consists of the loading step, the treatment step and the removal step, wherein the temperature of the superheated steam at the at least one steam inlet is constant throughout the treatment step.

Preferably, during the treatment step a temperature gradient is established across the treatment zone between the superheated steam at the at least one steam inlet and the steam at the at least one steam outlet. This is particularly relevant in continuous systems where the waste material is continuously flowed through the treatment zone. Preferably, the temperature difference between the superheated steam at the at least one inlet and the steam at the at least one steam outlet is at least 10° C., more preferable at least 50° C., more preferably at least 100° C. The temperature of the superheated steam at the at least one steam inlet is measured directly at the at least one steam inlet using any conventional means known in the art such as a thermocouple or thermometer. The temperature of the steam at the at least one steam outlet is measured directly at the at least one steam outlet using any conventional means known in the art such as a thermocouple or thermometer.

The method of steam generation used in the present invention is not particularly limited. The steam may be produced in any form of steam generator, for example the steam may be produced by a boiler or be derived from a steam waste source such as a power plant, steel manufacture or other industrial source of steam. Without being bound by any theory, for small scale, batch reactions it is likely that the steam will be derived from a small-scale boiler, whereas for larger scale continuous systems the steam may be derived from industrial sources.

The steam from the steam generator is heated in a superheater before being fed through the at least one inlet into the treatment vessel. Again, the type of superheater used is not particularly limited. Optionally, the superheater may comprise a heat exchanger, conduction heater or radiant heater.

Optionally, the apparatus further comprises a heat exchanger and the method further comprises the step of feeding the steam from the at least one steam outlet through the heat exchanger to recover residual heat from the steam.

### Continuous Process

Preferably, the method according to the present invention is a continuous process.

Preferably, during the treatment step the waste material is flowed in one direction through the treatment zone and the steam is flowed in the opposite direction through the treatment zone.

Without being bound by any theory flowing the waste material in one direction through the treatment zone and the steam in the opposite direction through the treatment zone creates a system where a temperature gradient is established over the treatment zone, meaning that as soon as the waste material enters a specific temperature zone the chemical components which will evaporate at that temperature will evaporate and then travel through the treatment zone to the at least one steam outlet. This means that fragile/volatile chemicals evaporate as soon as they reach the temperature zone at which they would evaporate and are not exposed to hotter portions of the treatment zone, which could lead to these fragile chemical moieties being degraded.

Flowing the waste through the treatment zone in one direction and flowing the steam through the treatment zone in the opposite direction can be thought of as a contraflow system, wherein the two reactants (that is waste material and steam) are being flowed in opposite directions.

Preferably, the treatment vessel used during the continuous process described above is a tubular treatment vessel. Optionally, the apparatus further comprises an auger feed configured to deliver the waste material into the treatment zone and a paddle stirrer configured to move the waste material through the treatment zone and to agitate the waste material during the treatment step. Optionally, the loading step comprises loading the waste material into the tubular treatment vessel and moving it into the treatment zone using the auger feed and the treatment step further comprises moving the waste material through the treatment zone using the paddle stirrer.

### Batch Process

The method according to the present invention may be a batch process.

Without wanting to be bound by any theory, it is believed that a batch process is particularly useful when low volumes of waste need to be processed. This might for example be for the extraction of specific chemicals from particular types of waste for example triethyl citrate from tyre waste and PVC.

In batch processes, it takes time for the superheated steam to penetrate the waste material, this means that the waste material is gradually heated to the temperature of the superheated steam at the at least one inlet and any chemicals formed are removed as the temperature rises.

Additionally, the method according to the present invention can be used to produce new (de-novo) chemicals which are useful in fields such as the pharmaceutical industry. Without being bound by any theory, it is believed that these chemicals are synthesised in the treatment zone as a result of the combination of radicals generated from the waste materials.

When the method of the present invention relates to a batch process the volume of the treatment vessel is preferably from about 0.001 to 0.75 m³, preferably from about 0.01 to 0.5 m³.

### Waste Material

The waste materials used in the method of the present invention are not particularly limited. Generally, it is envisaged that the waste material is a solid or liquid waste material. Without being bound by any theory, generally the term waste materials in the present invention excludes gaseous waste materials; although materials which are foams, or other liquid or solid mixtures comprising gases are not excluded from this definition.

Preferably, the waste material is a solid, a solid-slurry or a solid containing waste material. Generally, the waste material is derived from industrial, commercial and household activities.

Optionally, the waste material is an organic waste material, this refers to waste streams containing large amounts of carbon-based compounds found within natural, engineered, terrestrial and aquatic environments. Examples of organic waste materials include waste materials containing plastics and biological materials (such as plants and animal waste).

Optionally, the waste material is a mixed waste material, which is a combination of different types of waste, for example the waste material may be municipal solid waste which is a combination of different waste materials collected from households and may include items such as food waste, plastics, textiles, electronic waste, medical waste etc.

The waste material may be selected from the group consisting of municipal solid waste, agricultural waste, forestry waste, electronic waste, plastic waste, scrap tyres and tyre related waste, or a combination thereof.

Optionally, the waste is organic waste or industrial waste. Preferably, the waste is domestic organic waste, industrial waste or farm waste.

Agricultural waste is generally defined as any substance or object previously used in agriculture or horticulture. Agricultural waste may be selected from the group of straw, bagasse (sugar cane pulp), processing residues (such as those from pressing apples or from sugar beet), hay, silage, manure, empty pesticide containers, plastic silage wrappers, surplus milk, peat or a mixture of one or more of these components. A subset of agricultural waste may include waste derived from marine environments; this includes seaweed such as sargassum weed.

Forestry waste is generally defined as waste originating from the forestry industry. Forestry waste may be selected from the group of waste wood, wood chippings, saw dust, empty pesticide, fertilizer containers or a mixture of one or more of these components.

Electronic waste is generally defined as discarded electrical or electronic devices. The term electronic waste may refer to post consumer electronic waste or alternatively may refer to waste material produced in the production of electronic devices. These devices can be obtained from individual consumers or from commercial or business premises. Electronic waste may be selected from the group of fridges, freezers, cooling equipment, computers, telecommunications equipment, mobile phones, consumer electronic devices, solar panels, TVs, monitors, screens, LED bulbs, vending machines or a mixture of one or more of these components. In particular, electronic waste may comprise scrap solar panels, where the cost of separation by conventional means is considered prohibitive for recycling.

Optionally, the waste material is a plastic waste material. Optionally, the waste material is a mixed plastic waste material. Preferably, the waste plastic material comprises polyethylene terephthalate (PET), polyethylene (such as high-density polyethylene or low-density polyethylene), polyvinyl chloride (PVC), polypropylene (PP), polystyrene, polyurethanes, polyester, polyamide and acrylate-based polymers. More preferably, the waste plastic material comprises or consists of polyethylene or poly vinyl chloride (PVC) or a mixture of these two materials.

Optionally, the waste material is fibreglass, for example resin impregnated glass fibre. This may be present in items such as boats, wind turbine blades and car parts (e.g. body panels). In this case, the process according to the present invention may strip out the resin material leaving a loose mat of glass fibre.

Optionally, the waste material is scrap tyres or tyre related waste.

Optionally, the waste material may be hazardous waste.

Optionally, the particle size of the waste material is reduced from one average particle size to a smaller average particle size before being added to the treatment vessel. The particle size of the waste material may be reduced by shredding, grinding, pelletising, pulverising or comminuting the waste material. Optionally, the waste materials is shredded before being added to the treatment vessel. For example, the waste material may be shredded using an industrial shredder, such as the ZR2400H available from Untha shredding technology, UK. Optionally, the waste material before being added to the treatment vessel has an average (median) cross-sectional area of 10 cm³ or less, preferably 5 cm³ or less.

Optionally, at least 80% v/v of the waste material before being added to the treatment vessel in step (a) has a particle size of less than 37.5 mm as determined by sieve analysis using a British Standard test sieve shaker (Endecotts Ltd., London, UK).

The sieve shaker test may optionally be conducted as follows.
Use sieve apertures of 5, 6.7, 13.2, 20 and 37.5 mm and sieve diameters of around 70 cm, with a vertical separation between the sieves of approximately 15 cm;
Samples of 30 kg should be processed and the test sieve shaker is operated for a 20-minute period;
The particle size distribution can then be expressed either as the cumulative percent oversize in relation to the particle diameter.

Preferably, the waste material is substantially free of lime (calcium oxide and/or calcium hydroxide) and no lime is added to the treatment vessel during the treatment step. Without being bound by any theory, it is believed that there is no requirement to add lime to the waste materials because all of the chloride present in the waste materials is converted to HCl as a result of the method according to the present invention.

Generally, no additional catalysts are required for the operation of the present invention. Therefore, preferably, no additional catalysts are added to the waste material before treatment or during the treatment step.

### Pressure

The method according to the present invention is preferably conducted at nominal atmospheric pressure, since this avoids the need for pressure seals on the treatment vessel. Nominal atmospheric pressure may range from 50 to 200 kPa and is preferably from 80 to 120 kPa, more preferably about 100 kPa. Without being bound by any theory nominal atmospheric pressure is generally close to or the same as ambient atmospheric pressure.

In order to eliminate air from the treatment vessel the treatment vessel may be purged with steam prior to the reaction.

### Duration of Treatment

The duration of the treatment step according to the present invention is not particularly limited. Without being bound by any theory, it is believed that the duration of treatment is linked to the cross-sectional area of the waste material being treated, the type of waste material being treated, the volume of waste material in the treatment zone and the volume of superheated steam being applied to the treatment zone.

In a particular aspect, the duration of the treatment step is from 1 to 20 minutes, preferably from 1 to 10 minutes, more preferably from 1 to 5 minutes. The duration of the treatment step being defined as the total time a piece of waste material spends in the treatment zone. This may also be referred to as the residence time of the waste material in the treatment zone.

### Tyres

In a preferred aspect, the present invention relates to a method of treating waste tyres: that is the waste material is scrap tyres and/or tyre related waste. Tyre related waste relates to any tyre derived waste or waste derived from tyre manufacture. Generally, tyre related waste refers to natural or synthetically derived rubber waste and may include tyre sections, tyre treads, tyre inners and inner tubes.

Waste tyres are considered to be a hazardous form of waste and were banned from being disposed of in UK landfills in 2006. Waste tyres are inherently combustible and can cause harm to people and the environment by releasing chemicals and hazardous fumes when they are burnt. Piles of waste tyres can also provide nesting sites for vermin causing danger to human health.

Triethyl citrate is used as a plasticiser for polyvinyl chloride and tyres (often made from or containing butadiene rubber). Conventional energy recovery processes such as pyrolysis used to process waste tyres are not able to recover triethyl citrate from the waste tyres, meaning that this high value plasticizer is simply lost and cannot be recycled.

The present invention can allow the recycling of triethyl citrate from used tyres. The recycled triethyl citrate can be used as a plasticizer in new tyres and PVC materials.

The present invention may also allow the recycling of triethyl citrate from plastic materials other than tyres which also contain triethyl citrate.

The method according to the present invention may also allow the recycling or recovery of other additives or plasticisers added to tyres or plastics from used plastics or tyres. This may be in addition or as an alternative to the recycling of triethyl citrate.

Therefore, the method according to the present invention, may further comprise
condensing the steam from the at least one steam outlet to give a liquid reaction product;
separating an oily product from the liquid reaction product and
optionally, purifying the oily product to give a product comprising at least 80% triethyl citrate.

The method according to the present invention may also lead to the production of a solid reaction product, which freezes/crystalises out of the liquid reaction product. The solid material can then be separated from the liquid reaction product, for example by filtration.

The method of separating the oily reaction product form the liquid reaction product is not particularly limited. Optionally, the separation step may involve evaporating the aqueous component, for example by heating the aqueous reaction product at temperatures of around 100° C. Alternatively, the oily product could be separated from the aqueous component in the liquid reaction product by passing the mixture over a molecular sieve.

The method described above for the processing of tyres (and the recycling of triethyl citrate) may also be applied to waste materials comprising PVC and polyethylene.

Preferably, the method according to the present invention is a method of treating waste material using superheated steam in an apparatus, wherein the waste material is scrap tyres and/or tyre related waste, waste PVC or waste polyethylene,
the apparatus comprising:
   a treatment vessel comprising a treatment zone
   wherein at least one steam inlet is located at one end of the treatment zone and at least one steam outlet is located at the opposite end of the treatment zone;
   the method comprising:
a. a loading step, comprising loading the waste material at a temperature of less than 50° C. into the treatment zone;
b. a treatment step comprising:
   i. feeding superheated steam at a temperature of from 300 to 800° C. into the treatment zone through the at least one steam inlet and
   ii. removing steam and any gaseous reaction products through the at least one steam outlet;
c. a removal step comprising removing any remaining solid product from the treatment vessel after the treatment step; and
d. condensing the steam from the at least one steam outlet to give a liquid reaction product;
e. separating an oily product from the liquid reaction product;
f. optionally purifying the oily product to give a product comprising at least 80% triethyl citrate
   wherein apart from the superheated steam, any additional heating applied in the treatment zone during the treatment step raises the temperature of the treatment zone by 100° C. or less.

The method described above may also be applicable more generally to additives or plasticisers other than triethyl citrate added to tyres of plastics.

Preferably, the method according to the present invention is a method of treating waste material using superheated steam in an apparatus, wherein the waste material is scrap tyres and/or tyre related waste,
the apparatus comprising:
   a treatment vessel comprising a treatment zone
   wherein at least one steam inlet is located at one end of the treatment zone and at least one steam outlet is located at the opposite end of the treatment zone;
   the method comprising:
a. a loading step, comprising loading the waste material at a temperature of less than 50° C. into the treatment zone;
b. a treatment step comprising:
   i. feeding superheated steam at a temperature of from 300 to 800° C. into the treatment zone through the at least one steam inlet and
   ii. removing steam and any gaseous reaction products through the at least one steam outlet;
c. a removal step comprising removing any remaining solid product from the treatment vessel after the treatment step; and
d. condensing the steam from the at least one steam outlet to give a liquid reaction product;
e. separating an oily product from the liquid reaction product;
f. optionally purifying the oily product to give a product comprising at least 80% triethyl citrate
   wherein apart from the superheated steam, any additional heating applied in the treatment zone during the treatment step raises the temperature of the treatment zone by 100° C. or less.

### Electronic Waste Processing

In a further preferred aspect, the present invention relates to a method of treating electronic waste, that is the method according to the present invention where the waste material is electronic waste.

Electronic waste may be considered hazardous due to large quantities of harmful materials such as lead, cadmium, beryllium, or brominated flame retardants. Electronic waste also presents challenges from a recycling perspective with circuit boards containing precious metals such as gold, silver and platinum, as well as base metals such as copper, iron and aluminium being particularly difficult to recycle. It is estimated that currently between 10% and 20% of electronic waste is currently recycled due to the difficulty of the initial separation of the various components.

The method according to the present invention allows for the efficient treatment of electronic waste converting the electronic waste into a solid material which can be treated to easily remove a concentrate of metals (along with any ceramic components and/or semiconductors).

Consequently, in the method according to the present invention the waste material may be electronic waste and the method may further comprise the step of treating the solid product from step (c) to remove any carbonaceous material and fibre glass in order to give a concentrate of metals, ceramics and/or semiconductors. Optionally, the solid product from step (c) is treated in a cyclone or with a blast of gas (e.g. air or inert gas, such as N₂) to remove any carbonaceous material and fibre glass in order to give a concentrate of metals, ceramics and/or semiconductors. Optionally, the solid product from step (c) is treated with water to cool the solid product and remove the carbonaceous material.

Preferably, when electronic waste is being treated higher temperatures for example above 650° C. are used. As set out above, this leads to the carbon in the solid product reacting with the steam to form hydrogen and carbon monoxide, leaving behind a mixture of metals which can then be purified and re-used.

When the solid product is treated in a cyclone the heavy metal particles can be collected, whilst the lighter carbonaceous and fibreglass particles will remain airborne. Customised cyclones may be obtained from Sicca Dania A/S, Denmark.

The concentrate of metals, ceramics and/or semiconductors can be reprocessed using standard methods known in the art.

Preferably, the method according to the present invention is a method of treating waste material using superheated steam in an apparatus, wherein the waste material is electronic waste
the apparatus comprising:
   a treatment vessel comprising a treatment zone
   wherein at least one steam inlet is located at one end of the treatment zone and at least one steam outlet is located at the opposite end of the treatment zone;
   the method comprising:
a. a loading step, comprising loading the waste material at a temperature of less than 50° C. into the treatment zone;
b. a treatment step comprising:
   i. feeding superheated steam at a temperature of from 300 to 800° C. into the treatment zone through the at least one steam inlet and
   ii. removing steam and any gaseous reaction products through the at least one steam outlet; and
c. a removal step comprising removing any remaining solid product from the treatment vessel after the treatment step;
d. treating the solid product from step (c) to remove any carbonaceous material and fibre glass in order to give a concentrate of metals and/or ceramics;
   wherein apart from the superheated steam, any additional heating applied in the treatment zone during the treatment step raises the temperature of the treatment zone by 100° C. or less.

### Solid Product

The method according to the present invention results in the production of a solid product. The specific type of solid product obtained is not particularly limited and generally relates to the waste material used and the conditions during the treatment step.

Generally, the solid reaction product is a solid comprising elemental carbon. Optionally, the solid product has an elemental carbon content of at least 40% w/w. Optionally, the solid product has an elemental carbon content of at least 50% w/w, or at least 60% w/w, or at least 70% w/w. The elemental carbon content may be between 40% w/w and 90% w/w, or between 50% w/w and 80% w/w, or between 60% w/w and 70% w/w.

The present invention also relates to the solid product obtainable by the method according to the present invention.

As stated above, this solid product may contain high levels of elemental carbon and has a variety of uses.

The solid product may be used as a solid fuel, a close equivalent to a high-quality coal. This may be used to provide energy for industrial processes, or industrial energy generation or in domestic settings as a replacement for charcoal. Alternatively, this material could be used in other well-established coal-based technologies including hydrogen manufacture and “coal to oil technology”. The solid product could also be used as a replacement for coke in the steel industry.

Provided that the waste material was free of toxic contaminants, the solid product may also be used to enrich soils, which can benefit from the addition of elemental carbon. This considerably improves the soil condition and helps to retain moisture and nutrients. Furthermore, all of the minerals such as potassium, phosphate and magnesium from the waste material are retained in the solid product, reducing the amount of new fertilizer required when this material is added to soil

### Liquid Product

The method according to the present invention can be used to remove volatile components from the waste material. The method according to the present invention can also be used to synthesise new chemicals which can then be condensed from the steam at the at least one steam outlet. These products could be easily processed into feedstocks for the manufacture of a wide variety of chemicals and plastics.

In order to retain these products, it is necessary to separate them from the steam obtained at the outlet of the reaction vessel. Therefore, the method according to the present invention may further comprise the step of condensing the steam from the at least one steam outlet to give a liquid reaction product.

Preferably, the method according to the present invention is a method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
   b. a loading step, comprising loading the waste material at a temperature of less than 50° C. into the treatment zone;
   c. a treatment step comprising:
i. feeding superheated steam at a temperature of from 300 to 800° C. into the treatment zone through the at least one steam inlet and
ii. removing steam and any gaseous reaction products through the at least one steam outlet;
   d. a removal step comprising removing any remaining solid product from the treatment vessel after the treatment step; and
   e. optionally, condensing the steam from the at least one steam outlet to give a liquid reaction product;
   wherein apart from the superheated steam, any additional heating applied in the treatment zone during the treatment step raises the temperature of the treatment zone by 100° C. or less.

The present invention also relates to a liquid reaction product obtainable by a method according to the present invention.

The liquid products obtainable from the method according to the present invention may comprise phenolic acids, saccharides and short chain organics. In particular, the process according to the present invention leads to the production of high levels of cyclic compounds, which can optionally be converted to nitrophenols by reaction with nitric acid.

The liquid reaction product may also contain solids dissolved and/or dispersed therein. The liquid reaction product may be free of solids dispersed and/or dissolved therein.

The liquid reaction product may contain components which crystallise or solidify from the liquid over time and/or upon cooling to provide further solid materials that can be separated from the liquid product using known procedures, e.g. filtration. Either or both of this solid product and the remaining liquid may be desirable products.

The liquid reaction product can be separated into individual chemicals using industrial HPLC or other methods known to a person skilled in the art.

### Gaseous Product

The present invention may also result in the production of gaseous reaction products, these may be separated from the steam using known techniques, such as pressure swing adsorption, vacuum swing adsorption, temperature swing adsorption, by condensing the steam or by using membranes to purify the gases of interest.

The present invention also relates to a gaseous reaction product obtainable by the method according to the present invention.

The present invention also relates to a product which is a mixture of steam and the gaseous reaction product obtained at the steam outlet, obtained from a method according to the present invention.

### Apparatus

The present invention also relates to an apparatus for carrying out the method described above.

Generally, the apparatus comprises
a treatment vessel with a treatment zone,
a boiler for generating steam;
a superheater comprising a heat exchanger, conduction heater, or radiant heater for superheating the steam from the boiler;
a line connecting the superheater to the steam inlet;
   wherein, at least one steam inlet and at least one steam outlet are located at opposite ends of the treatment zone.

Preferably, the treatment vessel is a tubular treatment vessel.

Preferably, the apparatus further comprises a thermally insulating jacket at least partially surrounding the tubular treatment vessel.

Preferably, the apparatus comprises:
a tubular treatment vessel with a treatment zone,
a boiler for generating steam;
a superheater comprising a heat exchanger, conduction heater, or radiant heater for superheating the steam from the boiler;
a line connecting the superheater to the steam inlet;
a thermally insulating jacket at least partially surrounding the tubular treatment vessel;
   wherein, at least one steam inlet and at least one steam outlet are located at opposite ends of the treatment zone.

Preferably, the apparatus refers to an apparatus for use in a continuous process.

Optionally, the apparatus further comprises
a waste delivery system for delivering waste material into the tubular treatment vessel;
a means for conveying the waste material through the treatment zone.

Optionally, the waste delivery system comprises an auger feed, a gravity driven system, a conveyor system or a fluidised bed system. Preferably, the waste delivery system comprises an auger feed for delivering waste material into the tubular treatment vessel.

Optionally, the means for conveying the waste material through the treatment zone comprises an auger feed, a conveyor system, a ribbon mixer or a horizontal paddle stirrer. Preferably, the means for conveying the waste material through the treatment zone comprises a paddle stirrer.

Optionally, the apparatus comprises a treatment vessel, wherein the treatment vessel is a tubular treatment vessel. The tubular treatment vessel has a central axial paddle stirrer which is held in position by a bearing and driven by a motor at one end of the reaction vessel. Optionally, the reaction vessel also comprises a feed system which consists of a feed vessel with a central axial auger feed which is held in position by a bearing and is driven at one end by a motor. At the opposite end the feed vessel is connected to the reaction vessel. The feed system also incorporates a hopper which is suitable for dispensing the waste material into the feed vessel. The treatment vessel also has at least one steam inlet at one side of the treatment zone and at least one steam outlet at the opposite side of the treatment zone. The apparatus further comprises a boiler for generating steam, which is connected by a transfer line to a superheater comprising a heat exchanger, conduction heater or radiant heater for superheating the steam from the boiler. The apparatus further comprises a transfer line connecting the superheater to the at least one steam inlet.

Optionally, the apparatus may also comprise a mechanism for condensing any liquid products from the steam released from the steam outlet.

Optionally, the apparatus may further comprise a quenching system, such as a quench water spray device for quenching/cooling the solid product after it is removed from the treatment zone by the paddle stirrer.

Optionally, the apparatus further comprises a trap system for removing the solid product from the treatment vessel.

### Preferred Embodiments

Particularly preferred embodiments include:

A method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
   a. a loading step, comprising loading the waste material at a temperature of less than 50° C. into the treatment zone;
   b. a treatment step comprising:
i. feeding superheated steam at a temperature of from 300 to 800° C. into the treatment zone through the at least one steam inlet and
ii. removing steam and any gaseous reaction products through the at least one steam outlet; and
   c. a removal step comprising removing any remaining solid product from the treatment vessel after the treatment step
wherein apart from the superheated steam, any additional heating applied in the treatment zone during the treatment step raises the temperature of the treatment zone by 100° C. or less; and
wherein the method is a continuous process wherein during the treatment step the waste material is flowed in one direction through the treatment zone and the steam is flowed in the opposite direction through the treatment zone.

Preferably, the temperature of the superheated steam at the at least one inlet is from 400 to 550° C.

Preferably, the treatment vessel is a tubular treatment vessel and the apparatus further comprises
an auger feed configured to deliver the waste material into the treatment zone and
a paddle stirrer configured to move the waste material through the treatment zone and to agitate the waste during the treatment step;
   wherein
the loading step, comprises loading the waste material into the tubular treatment vessel and moving it into the treatment zone using the auger feed; and
the treatment step further comprises moving the waste material through the treatment zone using the paddle stirrer.

In a further particularly preferred embodiment, the present invention relates to a method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
   a. a loading step, comprising loading the waste material at a temperature of less than 50° C. into the treatment zone;
   b. a treatment step comprising:
i. feeding superheated steam at a temperature of from 300 to 800° C. into the treatment zone through the at least one steam inlet and
ii. removing steam and any gaseous reaction products through the at least one steam outlet; and
   c. a removal step comprising removing any remaining solid product from the treatment vessel after the treatment step
wherein apart from the superheated steam, any additional heating applied in the treatment zone during the treatment step raises the temperature of the treatment zone by 100° C. or less,
wherein the waste material is scrap tyres and/or tyre related waste and wherein the method further comprises:
   condensing the steam from the at least one steam outlet to give a liquid reaction product;
   separating an oily product from the liquid reaction product;
   optionally purifying the oily product to give a product comprising at least 80% triethyl citrate.

Preferably, the temperature of the superheated steam at the at least one inlet is from 400 to 550° C.

### BRIEF DESCRIPTION OF THE FIGURES

Embodiments of the invention will now be described, by way of example only, with reference to the accompanying drawings in which:

FIG. 1 is a diagram of apparatus suitable for carrying out the steam reforming process in batch mode.

FIG. 2 is a diagram of apparatus suitable for carrying out the steam reforming process in continuous mode

### DETAILED DESCRIPTION

The present invention will now be described in detail with reference to preferred embodiments and other optional features.

Unless defined otherwise, all technical and scientific terms used herein have the same meaning as commonly understood by one of ordinary skill in the art to which the invention pertains. Although, any methods and materials similar or equivalent to those described herein can be used in practice for testing of the present invention, the preferred materials and methods are described herein. In describing and claiming the present invention, the following terminology will be used in accordance with the definitions set out below. Unless clearly indicated otherwise, use of the terms “a,” “an,” and the like refers to one or more.

While the invention is described in conjunction with the exemplary embodiments described below, many equivalent modifications and variations will be apparent to those skilled in the art when given this disclosure. Accordingly, the exemplary embodiments set forth herein are considered to be illustrative and not limiting. Various changes may be made without departing from the scope of the invention which is defined by the claims. All references referred to herein are hereby incorporated by reference.

Each and every compatible combination of the embodiments described herein is explicitly disclosed herein, as if each and every combination was individually and explicitly recited. Additionally, where used herein, “and/or” is to be taken as a specific disclosure of each of the two specified features with or without the other.

Unless context dictated otherwise, the descriptions and definitions of the features set out herein are not limited to any particular aspect or embodiment and apply equally to all aspects and embodiments which are described where appropriate.

Where values are described as “at most” or “at least” it is understood that any of these values can be independently combined to produce a range.

Unless indicated otherwise, values provided are generally recorded at room temperature, that is, within the range 20-30° C. for example 20° C.

Where non-SI units are provided, it will be understood that these can be converted easily into SI units by the skilled person.

Where not otherwise specified percentage values refer to percentage determined on a weight per weight basis (w/w).

The use of headings herein is intended to be to assist the understanding of the invention by the reader and does not imply any limitation on the invention as defined in the claims.

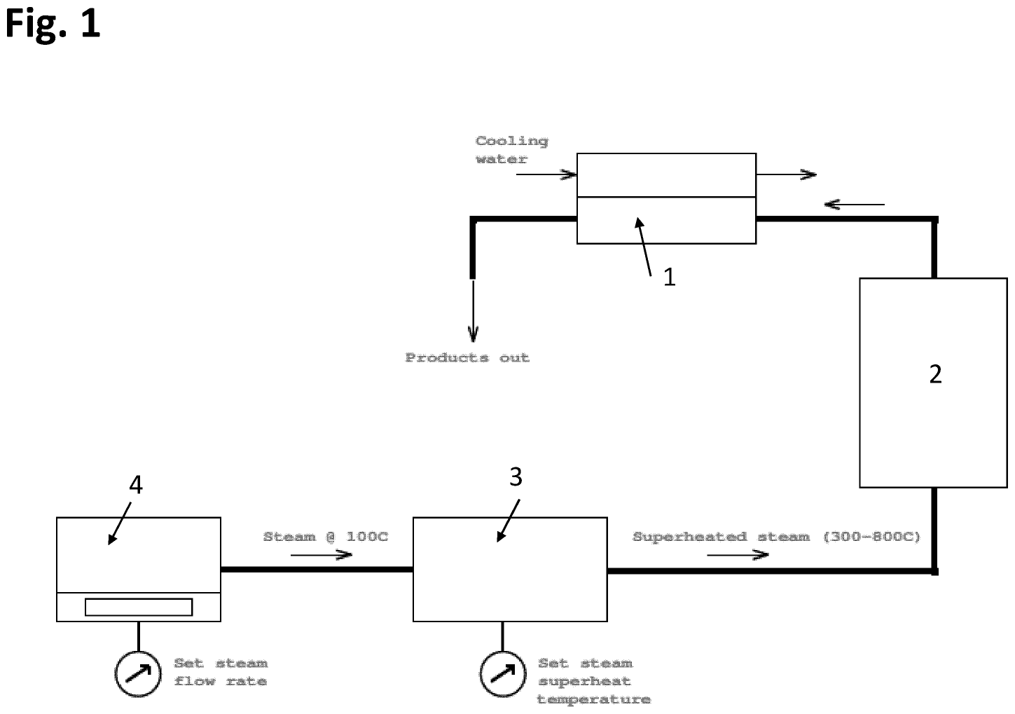

FIG. 1 is a diagram showing a typical configuration for a batch system according to the present invention. The apparatus in FIG. 1 consists of a treatment vessel (reaction vessel) 1 with a steam inlet on one side of the treatment vessel and a steam outlet on the opposite side. The steam inlet is connected to a steam generator 4 (e.g. a boiler or waste steam source) and superheater 3 for producing superheated steam. The steam outlet is connected to a heat exchanger 1.

To use the equipment, waste material is loaded into the reaction vessel. Steam at 100° C. is generated in the steam generator and then superheated to temperatures of 300 to 800° C. in the superheater. The superheated steam is then fed into the treatment vessel through the steam inlet and flows through the waste material. The steam is removed at the steam outlet and is flowed through the heat exchanger where it is cooled with liquid water (or other cooled using another cooling method well known in the art) causing liquid products to condense out of the mixture of steam and volatile reactants.

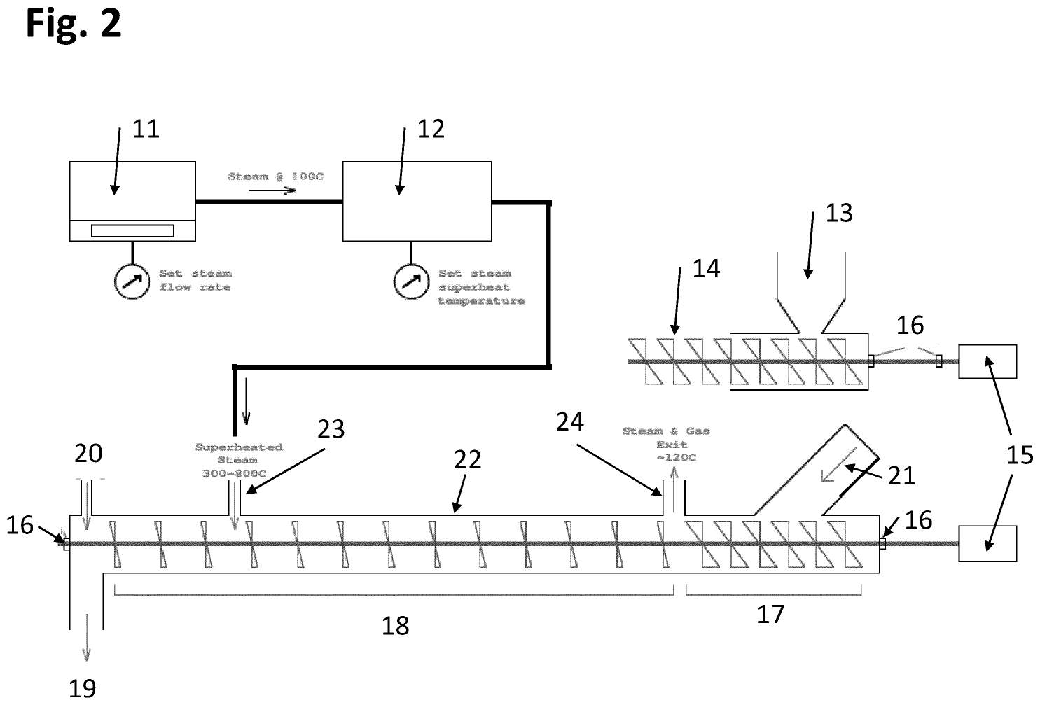

FIG. 2 is a diagram showing a typical configuration for a continuous system according to the present invention. The apparatus in FIG. 2 comprises a treatment vessel 22 having a central axial paddle stirrer 18 which is held in position by bearings 16 and driven by a motor 15 at one end of the reaction vessel. The reaction vessel also comprises a feed system 21 which consists of a feed vessel with a central axial auger feed 14 which is held in position by a bearing 16 and is driven at one end by a motor 15. At the opposite end the feed vessel is connected to the reaction vessel. The feed system also incorporates a hopper 13 which is suitable for dispensing the waste material into the feed vessel.

The treatment vessel also has a steam inlet 23 and at one side of the treatment zone and a steam outlet 24 at the opposite side of the treatment zone. The steam inlet is connected to a steam generator 11 (e.g. a boiler or waste steam source) and super heater 12 for producing superheated steam.

The treatment vessel may also comprise a drive auger system 17 for transporting waste material into the treatment zone before the steam outlet 24. The treatment vessel may also incorporate a quenching system 20, such as a quench water sprayer for quenching/cooling the solid product at the end of the reaction and a trap system 19 for collecting the solid product.

To use the equipment, waste material is loaded into the hopper. The hopper dispenses the waste material into the feed vessel and the auger feed in the feed vessel transport the waste material into the treatment vessel. In the treatment vessel the waste material is conveyed through the treatment vessel by the paddle stirrer and is removed at the end of the treatment vessel using a trap system. Steam is generated in the steam generator and then superheated in the superheater. The steam is then fed into the steam inlet and travels through the treatment vessel in the opposite direction to the flow of waste material until it is removed at the steam outlet at the end of the treatment zone.

### EXAMPLES

### Apparatus

### Reaction Vessel

The reaction vessel was a custom-made stainless steel cylindrical vessel, with a length of 200 mm and a diameter of 100 mm, sealed at both ends with end plates and with a single steam inlet of 10 mm and outlet of 15 mm at each end. This was surrounded by ceramic-fibre insulation approximately 25 mm thick, secured in place by a light-gauge aluminium jacket.

### Steam Generator

The steam generator was custom built using 1.2 mm thick copper sheet with a 3 KW electric heating element and a nominal water capacity of 4 litres at a water level maintained using an all-stainless steel ball valve connected to the mains water. The evaporation rate was regulated using an SCR based phase angle power controller permitting linear adjustment of actual heater power from zero to 3 kW.

### Steam Superheater

The steam superheater was custom made using a 1.5 metre length of 10 mm standard copper tube coiled to 80 mm diameter. Two ceramic 500 W radiant heaters were positioned 25 mm away from the copper coil at opposite sides facing towards the copper coil. The whole unit was fully encased in an insulated 0.5 mm thick stainless steel cylindrical container externally insulated using ceramic-fibre insulation material secured in place by a light-gauge aluminium jacket. The temperature of the copper tube immediately downstream of the heat transfer region was monitored using a K-type thermocouple linked to a programmable PID type temperature controller and display whose output regulated the power fed to the radiant heating elements in burst mode via a solid-state relay so as to regulate the steam exit temperature to the programmed value.

### Hydrolysis Method

100 g of sample was loaded into the reaction vessel of the apparatus shown in FIG. 2.

The steam generator was turned on. Once the steam temperature entering the superheater reached 100° C. the superheater was turned on. The superheated steam is PID controlled and set at 450° C. (measured at the outlet of the superheater which is connected to the inlet leading to the reaction chamber).

The temperature at the steam outlet of the reaction vessel, was monitored as it steadily rose until it reached 350° C. For the samples tested in this section, the time taken to achieve a temperature of 350° C. was around 40 minutes.

The steam/biogas mixture was then condensed with a water-cooled heat exchanger and collected. Two litres of condensate were obtained. The biogas was burnt showing a blue flame confirming the presence of hydrogen.

The reaction vessel was left 2 hours to cool and opened. The solid product was obtained as residue in the chamber.

A 100 ml sample of the condensate was used for GC-MS and LC-MS analysis.

### Example 1

### Polyethylene LC-MS analysis

100 g of polyethylene obtained from used polytunnel polythene was treated in the hydrolysis process described above with a steam inlet temperature of 500° C. The aqueous fraction was condensed and then prepared for analysis using the following procedure.

### Sample Preparation

The samples were diluted 100-fold by adding 10 μL of the liquid in 990 μL 10% MeOH (MeOH, VWR, LC-MS grade).

### Chromatographic Separation and Detection

LC-MS analyses were performed using an Agilent QTOF 6545 with Jetstream ESI spray source coupled to an Agilent 1260 Infinity II Quat pump HPLC with 1260 autosampler, column oven compartment and variable wavelength detector (VWD). The MS was operated in separate injections in either positive or negative ionization mode with the gas temperature at 250° C., the drying gas at 12 L/min and the nebulizer gas at 45 psi (3.10 bar). The sheath gas temperature and flow were set to 350° C. and 12 L/min, respectively. The MS was calibrated using reference calibrant introduced from the independent ESI reference sprayer. The VCap, Fragmentor and Skimmer was set to 3500, 125 and 45 V respectively. The MS was operated in all-ions mode with 3 collision energy scan segments at 0, 20 and 40 eV. Chromatographic separation of a 5 μL sample injection was performed on a InfinityLab Poroshell 120 EC-C18 (3.0×50 mm, 2.7 tm) column using H₂0 (Merck, LC-MS grade) with 0.1% formic acid (FA, Fluka) v/v and methanol (MeOH, VWR, HiPerSolv) with 0.1% FA v/v as mobile phase A and B, respectively. The column was operated at flow rate of 0.4 mL/min at 50° C. starting with 5% mobile phase B, as set out in table A.

TABLE A
Mobile phase for Chromatic separation and detection

| Time (min) | Mobile phase B (%) |
|---|---|
| 0.0 | 5 |
| 1.0 | 20 |
| 4.0 | 45 |
| 4.5 | 95 |
| 6.5 | 95 |
| 7.0 | 5 |
| 8.0 | 95 |
| 10.0 | 95 |
| 10.5 | 5 |
| 12.5 | 5 |

The VWD was set to detect at 2S4 and 320 nm wavelengths at a frequency of 2.S Hz. Data processing was automated in Qual 10 with molecular feature extraction set to the most intense 20 compounds for [M+H], [M−H]− and [M+HCOO]− ions. The results were searched against a Metlin database (containing 80,0S8 compound entries) with a forward score of 2S and reverse score of 70, and mass tolerances within 5 ppm of the reference library matches.

The samples were transparent solutions with slight oil layer adhered to the side of the plastic container. These were further diluted 100-fold to ensure compatibility and concentration levels suitable with LC-MS conditions. The diluted samples were analysed using both positive-and negative-mode LC-UV-MS using a chromatographic runtime of 15 min with additional organic washing to limit carry-over between samples (see gradient conditions for more details).

### Results

The peaks mostly eluted within the first 15 min of the chromatogram, suggesting mostly polar and semi non-polar compounds were present in the samples. The UV correlated better with the nBPC suggesting a greater representation of chromaphoric anions, possibly phenolic and aromatic acids, were present in the samples.

The top 10 compounds in the samples (based on LC-MS peak area) are given in table B

TABLE B
Top 10 compounds using positive mode detection for polyethylene

| | Compound |
|---|---|
| 1 | Triethyl citrate |
| 2 | Unknown |
| 3 | Unknown |
| 4 | D-1-[(3-5. Carboxypropyl)amino]-1-deoxyfructose |
| 5 | N-(1-Deoxy 1-fructosyl)isoleucine |
| 6 | Ascladiol |
| 7 | Sibiricose A5 |
| 8 | Deoxyloganic acid tetraacetate |
| 9 | 3R,7R)1,3,7Octanetriol |
| 10 | Ascladiol |

Other compounds of specific interest produced from polyethylene were:
18-hydroxypregna-1,4,20-trien-3-one
Propafenone
2-phenyl-1,3-propanediyl monocarbamate

### Example 2

### Tyre Rubber LCMS Analysis

100 g of tyre rubber (bought on the open waste materials market, e.g. 1 ton sized dumpy bags from Waste Tyre Specialists UK) was treated in the hydrolysis process described above. The aqueous fraction was condensed and then prepared for analysis using the following procedure.

### Sample Preparation

Sample preparation was performed as described above for example 1.

### Results

The peaks mostly eluted within the first 15 min of the chromatogram, suggesting mostly polar and semi non-polar compounds were present in the samples. The UV correlated better with the nBPC suggesting a greater representation of chromaphoric anions, possibly phenolic and aromatic acids, were present in the samples.

The top 10 compounds in the samples (based on peak area) are given in table C

TABLE C
Top 10 compounds using positive mode detection for tyre rubber

| | Compound |
|---|---|
| 1 | Triethyl citrate |
| 2 | Unknown |
| 3 | N-Acetylpyrrolidine |
| 4 | 8-Isoquinoline methanamine |
| 5 | 1,8-Diazacyclotetradecane-2,9-dione |
| 6 | 8-Methyl-5-propyloctahydroindolizine |
| 7 | 5-Hepten-3-yn-1-ol, 2,2-dimethyl-7- |
| | [methyl(1-naphthalenylmethyl)amino]-E |
| 8 | Cyclohexylamine |
| 9 | D-1-[(3-Carboxypropyl)amino]-1-deoxyfructose |
| 10 | 1,2-dihydrostilbene |

Other compounds of specific interest produced from tyre rubber (using negative mode detection) were:
18-hydroxypregna-1,4,20-trien-3-one (CHEBI:186912);
Vanilpyruvic acid;
Piperic acid;
4,5-hydroxypropafenone;
5,3-(2-Furyl) acrolein

Other compounds of specific interest produced from tyre rubber (using positive mode detection) were:
Triethyl citrate;
8-Isoquinoline methanamine.

### Example 3

### PVC LCMS Analysis

100 g of PVC (waste PVC cable covers e.g. from Doncaster Cables UK) was treated in the hydrolysis process described above for polyethylene.

The aqueous fraction was condensed and then prepared for analysis using the same procedure as that described above for example 1.

### Sample Preparation

Sample preparation was performed as described above for example 1.

### Results

The peaks mostly eluted within the first 15 min of the chromatogram, suggesting mostly polar and semi non-polar compounds were present in the samples. The UV correlated better with the nBPC suggesting a greater representation of chromaphoric anions, possibly phenolic and aromatic acids, were present in the samples.

The top 10 compounds in the samples (based on peak area) are given in table D

TABLE D
Top 10 compounds using positive or
negative mode detection for PVC

| | Compound | |
|---|---|---|
| | Positive mode | Negative mode |
| 1 | 18-hydroxypregna- | 3,4-Methylenedioxybenzoic acid |
| | 1,4,20-trien-3-one | |
| 2 | 5-Hydroxypropafenone | 3-(2-Furanyl)-2-propenal |
| 3 | 3-(1-Hydroxymethyl-1- | 18-hydroxypregna-1,4,20- |
| | propenyl)pentanedioic acid | trien-3-one |
| 4 | 2-Isopropylmaleate | 5-Hydroxypropafenone |
| 5 | Esmeraldic acid | 3-(1-Hydroxymethyl-1- |
| | | propenyl)pentanedioic acid |
| 6 | 2-Phenyl-1,3-propanediyl | 2-Isopropylmaleate |
| | monocarbamate | |
| 7 | 2-Acetylfuran | Esmeraldic acid |
| 8 | 3-Methyl-2-oxovaleric acid | O-Desacetylcephalothin |
| 9 | 2,3-Dihydroxy-2- | 2-Phenyl-1,3-propanediyl |
| | methylbutanoic acid | monocarbamate |
| 10 | cis-2-Carboxycyclohexyl- | unknown |
| | acetic acid | |

Other compounds of specific interest produced from PVC were:
4,4, methylenedioxybenzoic acid (piperonylic acid);
18-hydroxypregna-1,4,20-trien-3-one (CHEBI:186912);
Hydroxy Propafenone;
Phthalic acid mono-2-ethylhexyl ester (MEHP).

### Overall Conclusion Examples 1-3

Overall, mostly small organic acids and phenolic acids were detected in the hydrolysed samples, with the majority being present in the tyre rubber sample (example 2).

### Example 4

### Tyre Rubber GC-MS Analysis

GC-MS analysis was used to determine representation of non-polar compounds in the oil fraction of the samples.

100 g of tyre rubber (bought on the open waste materials market, e.g. 1 ton sized dumpy bags from Waste Tyre Specialists UK) was treated in the hydrolysis process described above. The aqueous fraction was condensed and then prepared for analysis using the following procedure.

### Sample Preparation

Samples were prepared for analysis by performing a liquid-liquid extraction. Approximately 2 ml of sample was mixed with 2 ml n-Hexane (GC Grade). The samples were vortexed, and the top n-hexane layer was collected for analysis.

### Chromatographic Separation and Detection

A 8890 gas chromatography (GC, Agilent) system coupled with 5977B MSD (MS, Agilent) was used for the analysis. Split injections of 1 μL were performed, with a split ratio of 50:1 (split flow of 20 mL/min), using with a single taper, ultra-inert wool inlet liner (Agilent 5190-2293). The inlet was heated to 250° C. with 3 mL/min septum purge flow. An Agilent HP-5 MS (30 m, 0.25 mm, 0.25 μm) column was used with He (BOC, N5.5) as the carrier gas, at a constant flow of 1.0mL/min. The column oven gradient started at 70° C., held for 4 mins, then ramped at 10° C./min to 200° C., held for 3 min, with a total analysis time of 20 mins. The MSD transfer line was set at 250° C., MSD source at 230° C., and the MSD quad temperature was set to 150° C. After an initial solvent delay of 6.5 min the MSD detection was performed using full scan mode, over the range of 30-300 m/z, with a scan speed of 1562 μs, and a gain factor of 15. Data analysis was performed in the Agilent Qualitative Analysis v.10.0 and used the NIST 17 library to identify and confirm compounds through spectral matching.

The samples were transparent solutions with slight oil layer adhered to the side of the plastic container. Liquid-liquid extraction was performed to extract non-polar compounds to ensure compatibility with the GC-MS analysis.

The MS/MS data was extracted from the peaks and searched against a NIST 17 library to identify putative compounds. Compound hits scores represent the prediction confidence, with most confident forecasts being closer to 100.

### Results

Numerous organosulphur-and heterocyclic aromatic compounds were detected in the Tyre Rubber sample. Based on the peak areas the peak at 12.5 min, representing 1,2-enzisothiazole, was the major component. This compound is typically used as vulcanization accelerator during rubber production.

The top ten compounds produced are given in table E below

TABLE E
Top 10 compounds using GC-MS analysis for tyre rubber

| | Compound |
|---|---|
| 1 | Unknown |
| 2 | Unknown |
| 3 | D-Limonene |
| 4 | 1,2-Benzisothiazole |
| 5 | Phenol, 2-methyl-5-(1-methylethyl) |
| 6 | Benzothiazole, 2-methyl- |
| 7 | 2,4,8-Trimethyl-1,2,3,4-tetrahydroquinoline |
| 8 | Quinoline, 1,2-dihydro-2,2,4-trimethyl |
| 9 | Quinoline, 2,4-dimethyl |
| 10 | Unknown |

### Example 5

### PVC GC-MS Analysis

100 g of PVC (waste PVC cable covers e.g. from Doncaster Cables UK) was treated in the hydrolysis process described above. The aqueous fraction was condensed and then prepared for analysis using the following procedure.

### Sample Preparation

Sample preparation was performed as described above for example 4.

### Results

The PVC samples had putative short chain alcohols, such as 2-ethyl-1-hexanol and 3-methyl-3-heptanol. Based on peak volumes the 8.8 min peak, representing 2-ethyl-2-hexanol, was the most prominent compound.

The top ten compounds produced are given in table F below

TABLE F
Top 10 compounds using GC-MS analysis for PVC

| | Compound |
|---|---|
| 1 | 3-Heptanol, 3-methyl |
| 2 | 1-Heptanol, 6-methyl |
| 3 | 1-Hexene, 3,5-dimethyl |
| 4 | (S)-3-Ethyl-4-methylpentanol |
| 5 | 1-Hexanol, 2-ethyl |
| 6 | Not determined |
| 7 | Not determined |
| 8 | Not determined |
| 9 | Not determined |
| 10 | Not determined |

### Example 6

### Wood Hydrolysis

100 g of wood (Chainsaw wood chippings obtained from felling mature ash and pine trees, with the chippings containing a mixture of ash and mixed pine wood with a moisture content of 20%) was treated in the hydrolysis process described above. The sample was treated for approximately 40 minutes and forty grams of carbon (charcoal) residue was obtained from the reaction chamber. The aqueous fraction was condensed and then prepared for analysis using the following procedure.

### Sample Preparation

The sample was diluted 100-fold by adding 10 μL of the yellow liquid in 990 μL 10% MeOH (MeOH, VWR, LC-MS grade).

### Chromatographic Separation and Detection

LC-MS analyses were performed using an Agilent QTOF 6545 with Jetstream ESI spray source coupled to an Agilent 1260 Infinity II Quat pump HPLC with 1260 autosampler, column oven compartment and variable wavelength detector (VWD). The MS was operated with separate sample injections for positive or negative ionization mode with the gas temperature at 250° C., the drying gas at 12 L/min and the nebulizer gas at 45 psi (3.10 bar). The sheath gas temperature and flow were set to 350° C. and 12 L/min, respectively. The MS was calibrated using reference calibrant introduced from the independent ESI reference sprayer. The VCap, Fragmentor and Skimmer was set to 3500, 125 and 45 V respectively. The MS was operated in all-ions mode with 3 collision energy scan segments at 0, 20 and 40 eV. Chromatographic separation of a 5 L sample injection was performed on a InfinityLab Poroshell 120 EC-C18 (3.0×50 mm, 2.7 μm) column using H₂O (Merck, LC-MS grade) with 0.1% formic acid (FA, Fluka) v/v and methanol (MeOH, VWR, HiPerSolv) with 0.1% FA v/v as mobile phase A and B, respectively. The column was operated at a flow rate of 0.4 mL/min at 50° C. starting with 5% mobile phase B, as shown in table G.

TABLE G
Details of the mobile phase used

| Time (min) | Mobile phase B (%) |
|---|---|
| 0.0 | 5 |
| 1.0 | 20 |
| 4.0 | 45 |
| 4.5 | 95 |
| 6.5 | 95 |
| 7.0 | 5 |
| 8.0 | 95 |
| 10.0 | 95 |
| 10.5 | 5 |
| 18.5 | 5 |

The VWD was set to detect at 254 and 320 nm wavelengths and a frequency of 2.5 Hz. Data processing was automated using Qual 10 software package, with molecular feature extraction set to the most intense 20 compounds for [M+H]+, [MH]− and [M−EFICOO]− ions. The results were searched against a Metlin database (containing 80,058 compound entries) with a forward score of 25 and reverse score of 70, and mass tolerances within 5 ppm of the reference library matches.

### Results

The sample was a transparent, yellow solution, diluted 100-fold for analysis to ensure compatibility and concentration levels suitable with LC-MS conditions. The solution was analysed using both positive-and negative-mode LC-UV-MS using a chromatographic runtime of 18.5 min with additional organic washing to limit carry-over between samples (see gradient conditions for more details).

The chromatographic separation was performed on a reverse phase (C18 end-capped) column, and the early elution profiles suggested a greater proportion of polar molecules that do not undergo extensive interactions with the stationary phase, resulting in early elution. When the MS data is compared between the two ionisation modes it is evident that there is good correlation between the peak detected by UV and those ionised in the negative mode. This suggested a greater representation of acidic molecules, rather than bases typically preferentially ionised in positive mode.

A molecular feature extraction (MFE) workflow was used for an untargeted screening approach, in which precursor masses are verified for charge carrier type (ie. [M−H]− and [M−EFICOO]−) and checked for isotopic distribution (ie. “C abundance and spacing), before being matched to a curated database of over 80K entries. In cases where no database matches were found, formulae were predicted for the mass features.

The top 10 compounds in the samples (based on peak area) are given in table H.

TABLE H
Top 10 compounds for woodchip

Compound

| | Negative mode | Positive mode |
|---|---|---|
| 1 | Methyl 4-oxo-2-pentenoate | Unknown |
| 2 | 3-Dihydroxy-2-methylbutanoic acid | Unknown |
| 3 | m-Coumaric acid | Unknown |
| 4 | 3-Hydroxy-4-methoxyphenyllactic acid | Unknown |
| 5 | 4-methoxycinnamic acid | Unknown |
| 6 | alpha-Furyl methyl diketone | Unknown |
| 7 | 6-Acetyl-D-glucose | Unknown |
| 8 | (S)-5-Hydroxymethyl-2[5H]-furanone | Unknown |
| 9 | Dimethyl D-malate | Methiuron |
| 10 | 3z-hepten-2 5-dione | Ginkgotoxin |

Other compounds of specific interest produced from woodchip were:
Methoxy cinnamic acid;
Dimethyl maleate;
Dimethyl succinate;
Piperic acid;
Carpacin;
Diethyl L-tartrate;
Methyl acrylate;
2-methoxy-1,4-hydroquinone (MHQ);
Dehydronuciferine;
(S)-(-)-5-Hydroxymethyl-2 (5H).

### Conclusion

Several hundred predicted molecules were detected in the hydrolysed wood sample, ranging from phenolic acids and furyl/furanone compounds to saccharides.

Without being bound by any theory, it is believed that the results for woodchip are representative for other agricultural and forestry waste.

1. A method of treating waste material using superheated steam in an apparatus, the apparatus comprising:
a treatment vessel comprising a treatment zone
wherein at least one steam inlet is located at one end of the treatment zone and at least one steam outlet is located at the opposite end of the treatment zone;
the method comprising:
a. a loading step, comprising loading the waste material at a temperature of less than 50° C. into the treatment zone;
b. a treatment step comprising:
i. feeding superheated steam at a temperature of from 300 to 800° C. into the treatment zone through the at least one steam inlet and
ii. removing steam and any gaseous reaction products through the at least one steam outlet; and
c. a removal step comprising removing any remaining solid product from the treatment vessel after the treatment step
wherein apart from the superheated steam, any additional heating applied in the treatment zone during the treatment step raises the temperature of the treatment zone by 100° C. or less.

2. The method of claim 1, wherein the temperature of the superheated steam at the at least one inlet is from 400 to 600° C., preferably from 400 to 550° C.

3. The method according to claim 1, wherein the method is a continuous process.

4. The method according to claim 3, wherein during the treatment step the waste material is flowed in one direction through the treatment zone and the steam is flowed in the opposite direction through the treatment zone.

5. The method according to claim 4, wherein the treatment vessel is a tubular treatment vessel and wherein the apparatus further comprises
an auger feed configured to deliver the waste material into the treatment zone and
a paddle stirrer configured to move the waste material through the treatment zone and to agitate the waste during the treatment step;
wherein
the loading step, comprises loading the waste material into the tubular treatment vessel and moving it into the treatment zone using the auger feed; and
the treatment step further comprises moving the waste material through the treatment zone using the paddle stirrer.

6. The method according to claim 1, wherein the method is a batch process and optionally wherein the treatment vessel has a volume of from about 0.001 to 0.75 m³, preferably from about 0.01 to 0.5 m³.

7. The method of claim 1, wherein apart from the superheated steam, no additional heating is applied to the treatment zone during the treatment step.

8. The method of claim 1, wherein the pressure in the treatment zone is from 50 to 200 kPa, preferably from 50 to 100 kPa.

9. The method of claim 1, wherein the solid product has a carbon content of at least 40%.

10. The method of claim 1, wherein the duration of the treatment step is from 1 to 20 minutes, preferably from 1 to 10 minutes, more preferably from 1 to 5 minutes.

11. The method of claim 1, wherein at least 80% v/v of the waste material in step (a) has a particle size of less than 37.5 mm as determined by sieve analysis using a British Standard test sieve shaker.

12. The method of claim 1, wherein the waste material is a mixed waste material.

13. The method of claim 1, wherein the waste material is selected from the group consisting of municipal solid waste, agricultural waste, forestry waste, (post-consumer) electronic waste, plastic waste, scrap tyres and tyre related waste, or a combination thereof.

14. The method of claim 1, wherein the method further comprises the step of condensing the steam from the at least one steam outlet to give a liquid reaction product.

15. The method of claim 13, wherein the waste material is scrap tyres and/or tyre related waste.

16. The method of claim 15, wherein the method further comprises:
condensing the steam from the at least one steam outlet to give a liquid reaction product;
separating an oily product from the liquid reaction product;
optionally purifying the oily product to give a product comprising at least 80% triethyl citrate.

17. The method of claim 1,
wherein the waste material is electronic waste
and wherein the method optionally further comprises the step of treating the solid product from step (c) in a cyclone or with a blast of gas to remove any carbonaceous material and fibreglass to give a concentrate of metals, ceramics and/or semiconductors.

18. The method according to claim 1, wherein the method consists of the loading step, the treatment step and the removal step and wherein the temperature of the steam at the steam inlet is constant throughout the treatment step.

19. The method according to claim 1, wherein the temperature difference between the superheated steam at the inlet and the steam at the steam outlet is at least 10° C., preferable at least 50° C., more preferably at least 100° C.

20. The method according to claim 1, wherein the waste material is substantially free of lime and wherein no lime is added to the treatment vessel during the treatment step.

21. The method according to claim 1, wherein the apparatus further comprises a heat exchanger and the method comprises the step of feeding the steam from the at least one steam outlet through the heat exchanger to recover residual heat from the steam.

22. A solid product obtainable by a method according to claim 1.

23. A product which is a mixture of stream and gaseous reaction product obtainable by a method according to claim 1.

24. A gaseous reaction product obtainable by a method according to claim 1.

25. A liquid reaction product obtainable by a method according to claim 14.

26. Apparatus for carrying out a method according to claim 1, comprising:
a treatment vessel with a treatment zone,
a boiler for generating steam;
a superheater comprising a heat exchanger, conduction heater, or radiant heater for superheating the steam from the boiler;
a line connecting the superheater to the steam inlet;
wherein, at least one steam inlet and at least one steam outlet are located at opposite ends of the treatment zone.
